(19) United States
(12) Patent Application Publication
(10) Pub. No.: US 2024/0324989 A1
(43) Pub. Date: Oct. 3, 2024

(54) SYSTEM FOR HANDS-FREE SECUREMENT OF AN ULTRASOUND TRANSDUCER

(71) Applicant: Rivanna Medical, Inc., Charlottesville, VA (US)

(72) Inventors: Zachary Leonard, Charlottesville, VA (US); Paul Sheeran, Waxhaw, NC (US); Kathryn Ozgun, Charlottesville, VA (US); Adam Dixon, Charlottesville, VA (US); Frank William Mauldin, JR., Troy, VA (US); Gerd Schmieta, Boston, MA (US); James Pelletier, Westhampton, MA (US)

(21) Appl. No.: 18/437,968

(22) Filed: Feb. 9, 2024

Related U.S. Application Data
(60) Provisional application No. 63/444,524, filed on Feb. 9, 2023.

(51) Int. Cl. A61B 8/00; A61B 8/08; A61B 46/10

(52) U.S. Cl. CPC A61B 8/4227; A61B 8/0841; A61B 8/4254; A61B 8/4281; A61B 8/4477; A61B 8/4488; A61B 8/461; A61B 8/56; A61B 46/10

(57) ABSTRACT

A system for affixing an ultrasound scanning transducer to a body for use in medicine during needle injection procedures.

### CROSS-REFERENCE TO RELATED APPLICATION

The present application relies on the disclosures of and claims priority to and the benefit of the filing date of the following U.S. Patent Application 63/444,524, filed Feb. 9, 2023. The disclosures of that application are hereby incorporated by reference herein in their entireties.

### STATEMENT REGARDING FEDERALLY SPONSORED RESEARCH

This invention was made with government support under Grant No. R44NS120798 awarded by the National Institutes of Health (NIH) National Institute of Neurological Disorders and Stroke (NINDS). The government has certain rights in the invention.

### TECHNICAL FIELD

The present invention is related to an apparatus that may be used to affix an ultrasound scanning transducer to a body for use in medicine during needle injection procedures.

### BACKGROUND OF THE INVENTION

Needle guidance procedures in medicine are numerous and comprise many different procedures including, for example, lumbar punctures, bone marrow biopsies, acute pain analgesia, and chronic pain therapy injections. The techniques available for injection guidance range from a palpation-based approach, where no image guidance is utilized, to guidance under an imaging modality such as ultrasound, computed tomography, or fluoroscopy. The palpation approach is low-cost and accessible at the bedside but suffers from low procedure success rates and higher rates of complications. Conventional ultrasound can improve success rates, and is utilized in some instances, but suffers from limitations including an extended learning curve and workflow barriers resulting from the need to simultaneously manipulate an ultrasound probe and insert a needle, the latter of which is typically a two-handed procedure. X-ray-based approaches, such as computed tomography or fluoroscopy, exhibit high success rates but expose the patient to ionizing radiation and increase procedure cost and are generally inaccessible at the bedside or incompatible with workflow constraints in fields such as emergency medicine.

To overcome the limitations of current state of the art approaches to medical needle guidance procedures, the present invention describes an apparatus that can be used to affix an ultrasound probe to a patient in order to facilitate interventional needle guidance workflow. The apparatus, which, in aspects, can be a single-use sterile consumable and support sterile procedures, can maintain hands-free imaging contact between the probe and the patient while providing minimally obstructed visual field and needle access to the patient anatomy relevant to the procedure such that the clinician may use one or both hands to place and advance the needle. This hands-free approach is an advancement compared to conventional ultrasound where the clinician requires one hand to hold the ultrasound imaging transducer and the second hand to advance the needle, or where an assistant is required to perform part of the procedure so that both hands are available to advance the needle. Various preferred embodiments of the invention are described herein.

### SUMMARY OF THE INVENTION

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

In embodiments, the present invention overcomes limitations of existing needle guidance systems by providing hands-free image guidance of needle advancement and providing a form factor compatible with sterile workflow that minimally obstructs the field of view and maximizes needle access. In embodiments, the invention interfaces to an ultrasound probe to stabilize the probe against patient anatomy during real-time imaging, allows for facile probe repositioning, and provides significant access around the perimeter of the probe for the physician to plan and execute the needle insertion. In addition, in aspects, the apparatus supports optional attachment of components to the ultrasound probe to guide the needle trajectory.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings, in which:

FIGS. 1A-1C are schematic illustrations of an exemplary apparatus that affixes to the patient anatomy and of an exemplary ultrasound probe incorporated into the apparatus.

FIG. 2 depicts the apparatus and an exemplary ultrasound probe affixed to the patient anatomy in an exemplary application.

FIG. 3 depicts a flow diagram of a process by which the present apparatus may be used by a clinician to assist in a needle guidance procedure.

FIGS. 4A-4C are schematic illustrations of an exemplary apparatus with rotational components and of an exemplary dual-array probe interfaced with the apparatus.

FIGS. 5A-5D are schematic illustrations of an exemplary apparatus that affixes to the patient anatomy and of an exemplary ultrasound probe incorporated into the apparatus. These schematic illustrations illustrate an apparatus comprised of two base components integrated into a sterile patient drape with elastic bands that secure the ultrasound probe between the two base components.

FIGS. 6A-6D are schematic illustrations of an exemplary apparatus with angulation set by the configuration of securing components that interface with the ultrasound probe.

FIGS. 7A-7B are schematic illustrations of an exemplary securing component that contains an acoustically transmissive material, an acoustic coupling dispensation component, and/or an acoustically transmissive adhesive component. The securing component can be integrated with other securing components, which may include elastic bands, and with a sterile probe drape or sheath.

FIG. 8 depicts an exploded view of an exemplary dual-array probe with a U-slot and needle guide insert.

FIG. 9 depicts an exemplary dual-array probe connected to an exemplary software platform and medical cart.

FIG. 10 depicts a flow diagram of a process by which the present apparatus with an exemplary dual-array probe with position tracking may be used by a clinician to assist in a needle guidance procedure.

### DETAILED DESCRIPTION OF VARIOUS EMBODIMENTS OF THE INVENTION

Ultrasound imaging transducer assemblies are used in a variety of medical or clinical applications to enable medical imaging functions. In this non-limiting example, an ultrasound imaging transducer is disposed within a transducer assembly to deliver a pulse, tone, sequence or programmed energy signal into a target location to be imaged. A specific example is one or more ultrasound transducer elements that deliver an ultrasound signal into a patient's body and detect a return signal so as to form a computer-generated image of the target region. Different ultrasound imaging modes can be utilized, depending on a given application and design as known to those skilled in the art. The present disclosure can be used in medical ultrasound applications but is not limited to this application. Those skilled in the art will appreciate that a variety of types of transducers, signal transmitters and/or receivers and other arrays can also benefit from the present invention, which are comprehended hereby. The preferred embodiments herein describe needle guidance. Those skilled in the art will appreciate that the present invention may be used to guide a variety of medical instruments including, but not limited to, a catheter, trocar, ablation instrument, or therapy applicator. The present invention can be utilized, in a preferred embodiment, with systems and methods previously disclosed by Mauldin et al. (PCT/US2019/012622), which is incorporated by reference herein, for automated three-dimensional detection, guidance, and visualization of ultrasound-based therapy guidance procedures.

In the embodiment of the present invention for medical applications of needle guidance described herein, an objective of the device is to stabilize the ultrasound probe against the patient anatomy such that the physician can remove their hands from the device while acoustic coupling is maintained for image guidance of needle insertion. In embodiments, additional objectives include minimal visual obstruction of the underlying anatomy relevant to the medical procedure, minimal obstruction of access to needle insertion points, (in aspects, simple) repositioning and reorientation of the ultrasound probe, and compatibility with a sterile workflow. In embodiments, the invention is comprised of one or more base component that can be removeably affixed to a patient near or adjacent to a patient anatomy relevant to the medical procedure. The base component may comprise rigid, semi-rigid, or substantially rigid materials, such as plastics or metals, and provides an anchor point to secure the ultrasound transducer in contact with the patient anatomy. In embodiments, the invention is comprised of one or more securing component that can be attached to the one or more base component and the ultrasound transducer and provides a directed force that maintains contact between the ultrasound transducer and the patient anatomy. The one or more securing component may comprise flexible or substantially flexible materials, such as rubber, some plastics, or fabric, or may comprise rigid, semi-rigid, or substantially rigid materials, such as plastics or metals. In embodiments, the invention provides a mechanism for adjusting a position of the ultrasound transducer affixed to the patient anatomy by the apparatus, which may comprise an arrangement of one or more base component and one or more securing component designed so that a user may manually reposition the ultrasound probe within the apparatus, such as by applying force to the ultrasound probe handle, one or more securing component, or one or more base component.

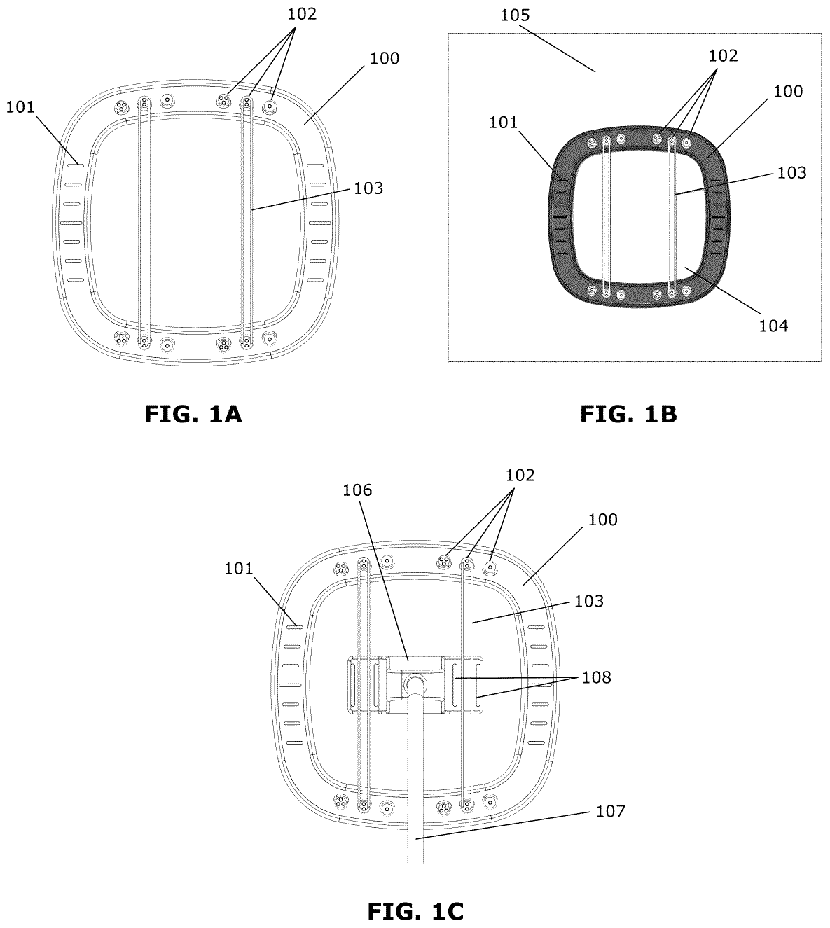

In an exemplary embodiment, an apparatus is depicted in FIG. 1A. The apparatus has a base component 100 that affixes to the patient anatomy. Non-limiting examples of the method of affixing the base component 100 to the patient anatomy include incorporation of an adhesive layer on the patient contact side, incorporation of straps around the patient anatomy, suction mechanisms, magnetic mechanisms or other approaches that would secure the position of 100 on the patient anatomy. In this non-limiting example, the base component is depicted to have rectangular shape with rounded corners, but other shapes are envisioned. The design may be of configurations that conform to specific patient anatomy and provide visual access and needle access, including but not limited to circular or semi-circular shaping. Fiducial markers 101 can provide measurements to the user for planning of the procedure and probe positioning. In aspects, a component 102 can be used to attach rubber bands 103 or straps along the perimeter of the apparatus that secure an ultrasound probe within the apparatus.

In a non-limiting embodiment depicted in FIG. 1B, the apparatus 100 may conform with a sterile workflow in which the apparatus is applied over a sterilized portion of the patient anatomy 104 and can be uncovered for imaging interrogation and needle insertion while a sterile drape 105 (e.g., procedural drape) that covers the non-sterile patient anatomy is incorporated with or into the outer edge of the apparatus 100. In embodiments, the drape can be of the same size and material used during epidural and spinal anesthesia, or other similar needle guidance, procedures.

In a non-limiting embodiment depicted in FIG. 1C, an ultrasound probe 106 and ultrasound cable 107 can be integrated into the device and can be retained within the securing bands 103 by positioning guides 108 that limit lateral movement. The positioning guides 108 can be incorporated directly into the ultrasound probe 106, or in other non-limiting embodiments may consist of sterile components that clip onto the ultrasound probe housing. In a non-limiting embodiment, an ultrasound signal cable 107 connects the ultrasound probe 106 to a computer processor, with alternative embodiments including wireless transmission of signals or incorporation of a computer processor and display directly into the probe housing.

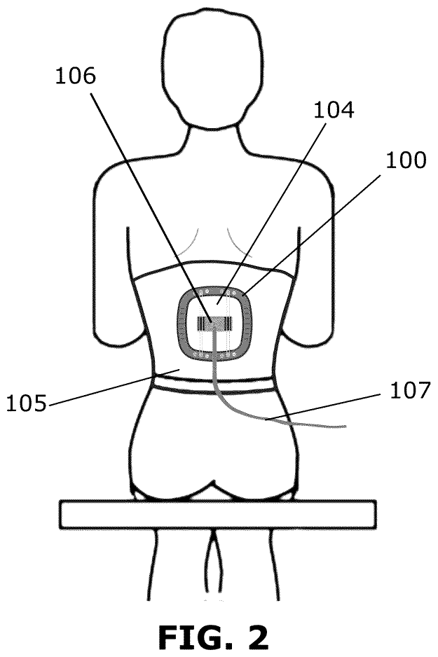

A schematic illustration of a procedure involving a patient's spinal anatomy is depicted in FIG. 2. In this non-limiting embodiment, the underside of the apparatus 100 contains medical-grade adhesive designed to affix the base component of the apparatus to the patient's skin, centered over the region the needle is to be inserted. After interrogating the sterilized anatomy with the ultrasound probe 106, the physician leaves the ultrasound probe in position and uses one or both hands to insert the needle within the “window” provided by the apparatus 100.

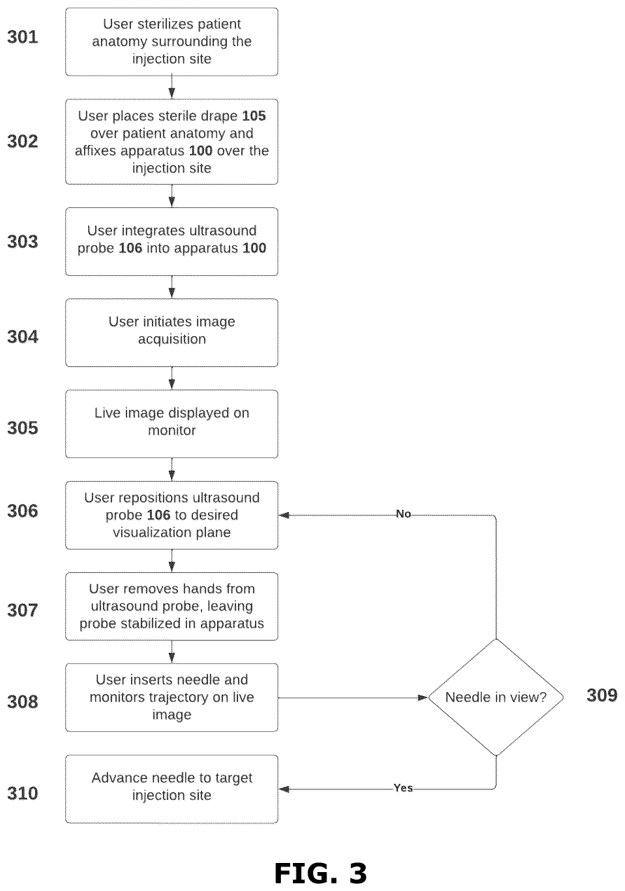

A flow diagram of an embodiment for the present invention used in a clinical needle guidance procedure is depicted in FIG. 3. At block 301, the user first sterilizes the patient anatomy around the intended injection site. At block 302, the user places the sterile drape component 105 of the apparatus over the non-sterile patient anatomy, leaving an open window over the sterilized anatomy. The base component of the apparatus 100 is then affixed to the patient anatomy by an adhesive layer incorporated into the component. At block 303 the ultrasound probe is integrated into the apparatus as depicted in FIG. 2 at the desired imaging position and coupled to the patient's body using ultrasound coupling gel or other lubricant compatible with medical ultrasound and known to those skilled in the art. Next, at block 304 an image acquisition is initiated by the user. At block 305, the ultrasound image acquisitions are transmitted to a computer system and the reconstructed image is displayed real-time on a monitor. At block 306, the user may translate the ultrasound probe 106 manually within the apparatus until the desired anatomy is within the imaging plane. At block 307, the user removes their hands from the ultrasound probe 106, leaving the probe secured in the apparatus 100 while continuing to display real-time anatomical images. In block 308, the user uses one or both hands to insert the needle and monitors the trajectory of the needle entering the real-time imaging display. At block 309, if the real-time imaging display indicates the needle is no longer in view, the user may reposition the ultrasound probe 106 within the apparatus 100 to regain visualization of the needle. At block 310, the user completes the procedure by advancing the needle to the target site, confirmed by real-time image display.

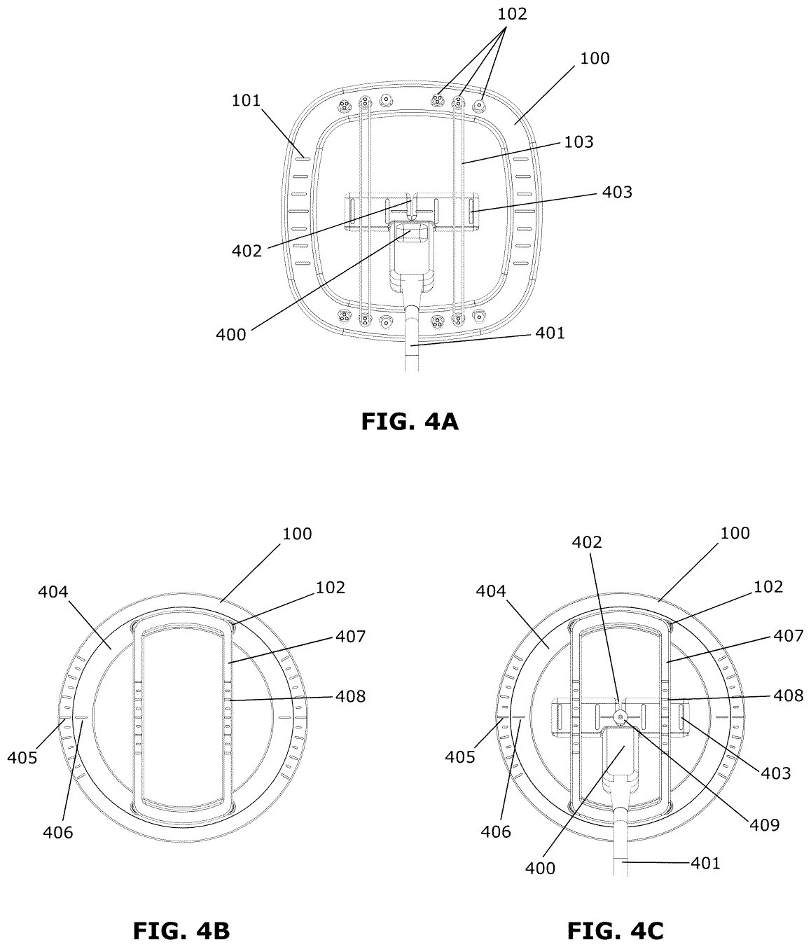

In a non-limiting embodiment depicted in FIG. 4A, a unique split-array ultrasound probe 400 with a U-slot 402 for facile in-plane needle access, the subject matter of U.S. patent application Ser. No. 17/950,399 (incorporated herein by reference), is integrated into or otherwise attached or connected to the previously described apparatus 100. The device is retained within the securing bands 103 by positioning guides 403 that limit lateral movement. The positioning guides 403 may be incorporated directly into the ultrasound probe 400, or in other non-limiting embodiments may consist of sterile components that clip onto the ultrasound probe housing. In a non-limiting embodiment, an ultrasound signal cable 401 connects the ultrasound probe 400 to a computer processor, though alternative embodiments may include wireless transmission of signals or incorporation of a computer processor and display directly into the probe housing. In a non-limiting embodiment, the interface between the ultrasound probe 400 and the ultrasound signal cable 401 is angled relative to the ultrasound probe body 400 as to minimize the vertical profile of the ultrasound probe relative to the patient anatomy, improve stability within the apparatus, and/or maximize procedure access around the base of the ultrasound probe 400. Non-limiting examples include a probe-cable interface that angles between 30 degrees and 90 degrees away from the probe body, a probe-cable interface with said angling that is centered or off-centered along the front or back of the ultrasound probe body, and a probe-cable interface with said angling that is centered or off-centered along either side of the probe body.

In a preferred embodiment, an apparatus is depicted in FIG. 4B. In this non-limiting example, the base component is circular, but may be designed in other configurations that conform to specific patient anatomy and provide visual access and needle access. A second component 404 provides a rotation ring by which the inner components of the apparatus can rotate to reposition the ultrasound probe while the base component 100 remains secured to the patient. Fiducial markers 405 and 406 provide indications of degree of rotation to the user. In a non-limiting embodiment, a position tracking clip 407 that secures the ultrasound probe within the apparatus is attached to a component 102 on the base apparatus. In a non-limiting embodiment, the position tracking clip 407 contains electrical components that enable determination of the probe position within the clip. Fiducial markers 408 can provide an indication of the position along the position tracking clip 407. Illustration of the ultrasound probe 400 incorporated into the apparatus is depicted in FIG. 4C. The ultrasound probe 400 can incorporate sensors that read the position of the probe within the position tracking clip 407 and transfer these signals to the imaging system through the ultrasound probe cable 401. In preferred embodiments, the position measurement is accomplished through magnetic/inductive or resistive methods. In one such embodiment, passive, non-self-powered elements of the sensor system are incorporated in the position tracking clip 407, while active powered sensors are incorporated in the ultrasound probe 400. In a non-limiting embodiment, a needle guide 409 supplied with the apparatus is inserted into the U-slot 402 of the ultrasound probe 400 to guide needle trajectory within the apparatus and facilitate in-plane needle delivery during the procedure.

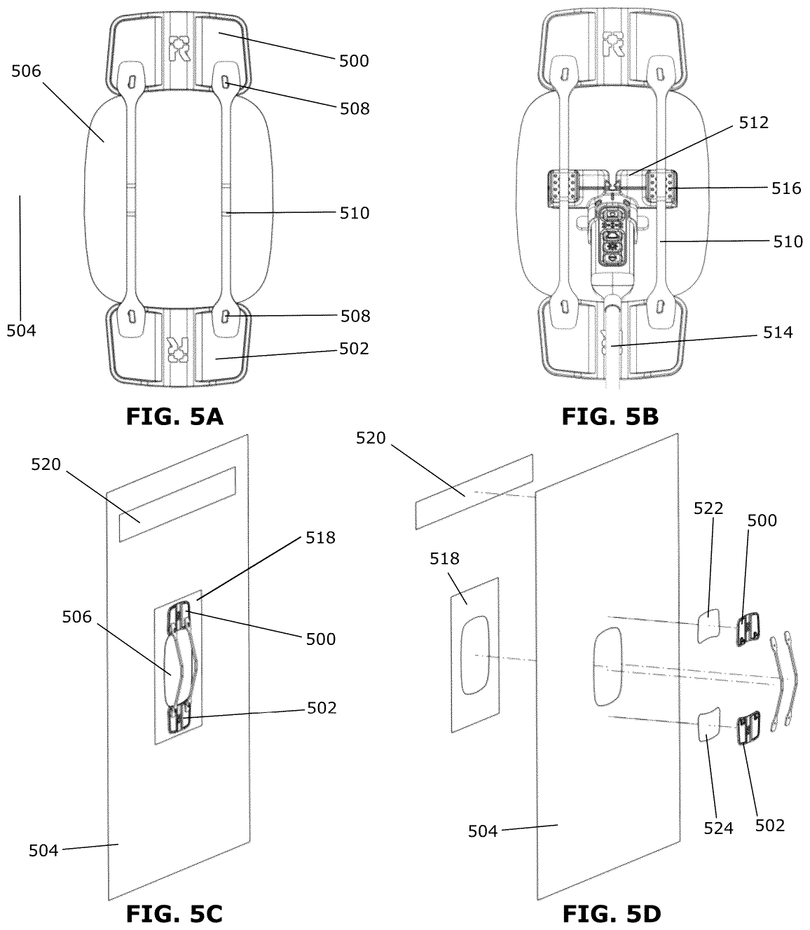

In an exemplary embodiment, an apparatus is depicted in FIG. 5A. The apparatus has two base components 500 and 502 that affix to a patient drape 504. Non-limiting examples of the method of affixing the base components 500 and 502 to the patient drape include incorporation of an adhesive layer on the patient contact side, suction mechanisms, magnetic mechanisms or other approaches that would affix the positions of 500 and 502 on the patient drape. In this non-limiting example, the base components are depicted to have a rounded shape that conform to the top and bottom positions of the patient drape opening 506, but other shapes and positions of incorporation along the patient drape opening 506 are envisioned. The design may be of configurations that conform to specific patient anatomy and provide visual access and needle access, including but not limited to circular or semi-circular shaping. Securing components 508 may be used to secure elastic bands 510, straps, or other flexible materials, connect the two base components 500 and 502 across the patient drape opening 506 for the purpose of securing an ultrasound transducer.

In a non-limiting embodiment depicted in FIG. 5B, an ultrasound probe 512 and ultrasound signal cable 514 can be integrated into the device and can be retained within the securing bands 510 by positioning guides 516 that limit lateral movement. The positioning guides 516 can be incorporated directly into the ultrasound probe 512, or in other non-limiting embodiments may consist of sterile components that clip onto the ultrasound probe housing, or may consist of sterile components attached to an ultrasound probe sheath that can be attached to the ultrasound probe housing. In a non-limiting embodiment, an ultrasound signal cable 514 connects the ultrasound probe 512 to a computer processor, with alternative embodiments including wireless transmission of signals or incorporation of a computer processor and display directly into the probe housing.

In a non-limiting embodiment depicted in FIG. 5C, the base components 500 and 502 may conform with a sterile workflow in which they are applied over a sterilized portion of the patient anatomy 506 and can be uncovered for imaging interrogation and needle insertion while a sterile drape 504 (e.g., procedural drape) that covers the non-sterile patient anatomy is incorporated with or into the base components 500 and 502. In embodiments, the drape can be of the same size and material used during epidural and spinal anesthesia, or other similar needle guidance, procedures. Affixing components 518 and 520 are incorporated into the patient drape for the purpose of securing the drape 504 to the patient anatomy. Non-limiting examples of the affixing components 518 and 520 include adhesive layers, straps, suction mechanisms, magnetic mechanisms or other approaches that would secure the position of the patient drape 504 on the patient anatomy.

In a non-limiting embodiment depicted in FIG. 5D, the base components 500 and 502 are attached to the patient drape 504 with affixing components 522 and 524, while the patient drape 504 is affixed to the patient anatomy with affixing components 518 and 520. Non-limiting examples of the affixing components 522 and 524 include adhesive layers, straps, suction mechanisms, magnetic mechanisms or other approaches that would secure the position of the patient drape 504 on the patient anatomy.

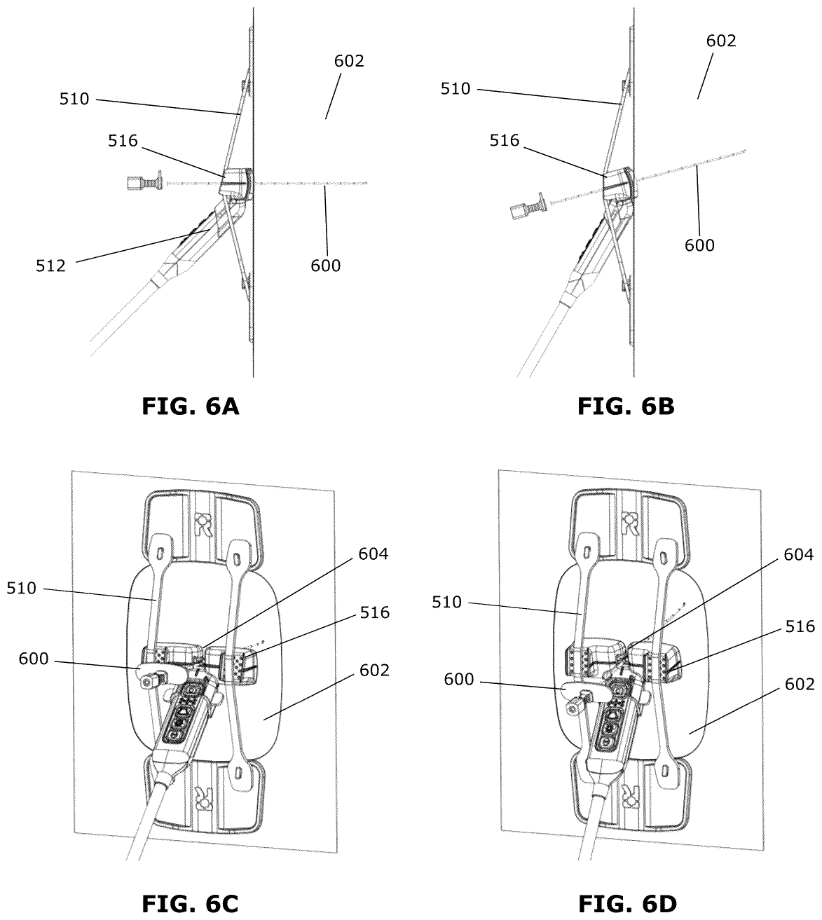

In a non-limiting embodiment depicted in FIG. 6A, a unique split-array ultrasound probe 512 with a slot for facile in-plane needle access, the subject matter of U.S. patent application Ser. No. 17/950,399, is secured within the apparatus using elastic bands 510 and configured, such as through manual manipulation, to hold an angulation that allows a straight angle of entry of a needle 600 relative to the patient anatomy 602. In FIG. 6B, the angulation of the ultrasound probe 512 within the positioning apparatus 516 is reconfigured, such as through manual manipulation, to provide an angled needle 600 entry relative to the patient anatomy 602. Non-limiting examples include an apparatus that allows a needle angulation of between 0 and 20 degrees relative to the straight angle of entry depicted in FIG. 6A. FIG. 6C depicts a front-facing view of the depiction in FIG. 6A, with the needle 600 placed in a U-slot 604 for facile in-plane needle access. FIG. 6D depicts a front-facing view of the depiction in FIG. 6B, in which the needle angulation has been adjusted by user manipulation and the securing components 510 and positioning components 516 stabilize the ultrasound probe against the patient anatomy 602.

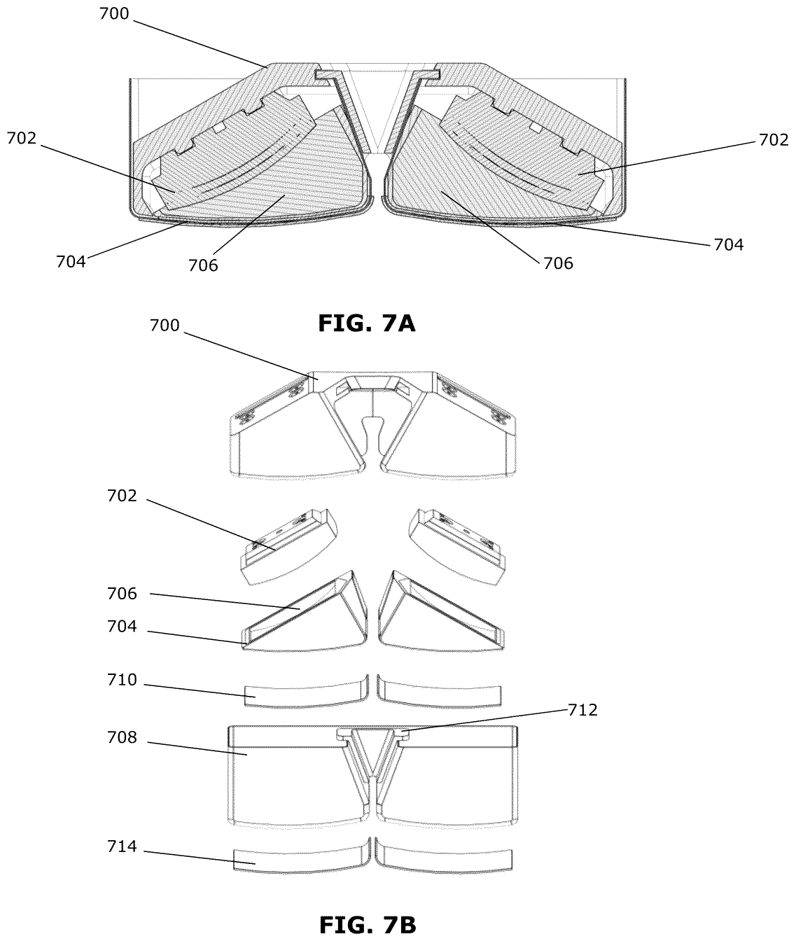

A component-level view of an exemplary securing component designed as a dual-array housing 700 is depicted in FIG. 7A. An exploded component-level view of the exemplary dual-array housing 700 is depicted in FIG. 7B. The imaging device can contain dual ultrasound arrays 702 and associated electromechanical components familiar to those skilled in the art which are packaged in mechanical housings. The imaging device can include acoustic lenses 704 and acoustic couplants 706, which may be discrete parts or integral to other mechanical housing(s), for optimized transmission of acoustic energy from the arrays 702. In a non-limiting embodiment, the lenses 704 and acoustic couplants 706 may comprise sterile single-use components that can be used to impose a sterile barrier between the ultrasound arrays 702 and patient anatomy. Additional ultrasound arrays, matrix transducer arrays, or C-MUT arrays may be used in place of the two ultrasound arrays 702 in order to improve field of view or image acquisition speeds. A sterile single-use assembly 708 may encapsulate the assembly of arrays 702, acoustic couplants 706, and lenses 704. In a non-limiting embodiment, the probe housing may comprise sterile single-use components that can be used to impose a sterile barrier between the ultrasound arrays 702 and patient anatomy. In a non-limiting embodiment, the sterile single-use assembly 708 may acoustically couple to the lenses 704 using an acoustic coupling component 710, which may comprise an adhesive film, an aqueous material, such as acoustic gel, an oil, or a sponge designed to retain and dispense an aqueous material or an oil. In a non-limiting embodiment, the probe housing 708 may comprise securing components attached to a sterile probe drape, such as through an adhesive material incorporated into the sterile single-use assembly 708. In a non-limiting embodiment, the sterile single-use assembly 708 may comprise a securing component 712 that is designed to secure the dual-array housing 700 within the sterile single-use assembly 708. In aspects, the disposable needle guide 409 inserts into the sterile single-use assembly 708 to provide either sterile or non-sterile needle trajectory guidance. In a non-limiting embodiment, the sterile single-use assembly 708 may comprise an acoustic coupling component 714 that provides an acoustically transmissive medium between the sterile single-use assembly 708 and the patient anatomy. The acoustically coupling component may comprise an adhesive film, an aqueous material, such as acoustic gel, an oil, or a sponge designed to retain and dispense an aqueous material or an oil.

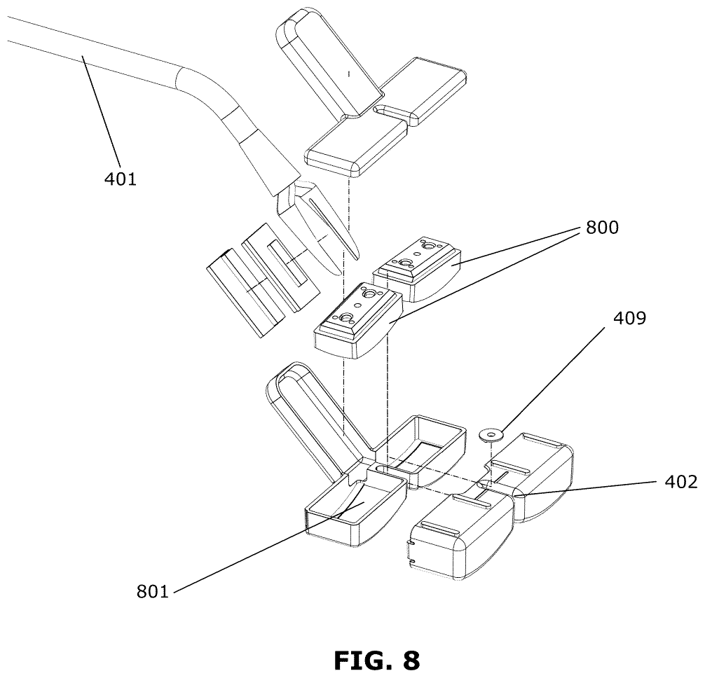

An exploded, component-level view of an exemplary dual-array ultrasound probe 400 is depicted in FIG. 8. The imaging device can contain dual ultrasound arrays 800 and associated electromechanical components familiar to those skilled in the art which are packaged in mechanical housings. Ultrasound probe 400 electronics may incorporate a position encoder, signals of which are relayed through the ultrasound probe cable 401 to a computer processor to instruct linear position changes to the linear actuator, image acquisition from the ultrasound arrays 800, and signal or image processing steps applied to the acquired ultrasound image signals. The imaging device can include acoustic lenses and acoustic couplants 801, which may be discrete parts or integral to other mechanical housing(s), for optimized transmission of acoustic energy from the arrays 800. In a non-limiting embodiment, the lenses and acoustic couplants 801 may comprise sterile single-use components that can be used to impose a sterile barrier between the ultrasound probe 400 and patient. Additional ultrasound arrays, matrix transducer arrays, or C-MUT arrays may be used in place of the two ultrasound arrays 800 in order to improve field of view or image acquisition speeds. In aspects, the disposable needle guide 409 inserts into the probe housing to provide either sterile or non-sterile needle trajectory guidance. The needle guide 409 can constrain the location of the needle at the base of the U-slot 402 to allow accurate needle placement in the desired anatomic location and within the ultrasound imaging plane. The needle guide 409 may allow removal of the needle from the anatomy and imaging device 400 through the length of the U-slot 402. The needle guide 409 can be configured to either constrain or allow removal of the needle through mechanisms including but not limited to rotation, opening, or removal of the needle guide. The entire ultrasound probe assembly may be covered by a sterile sheath before being incorporated into the stabilization apparatus to support sterile procedures.

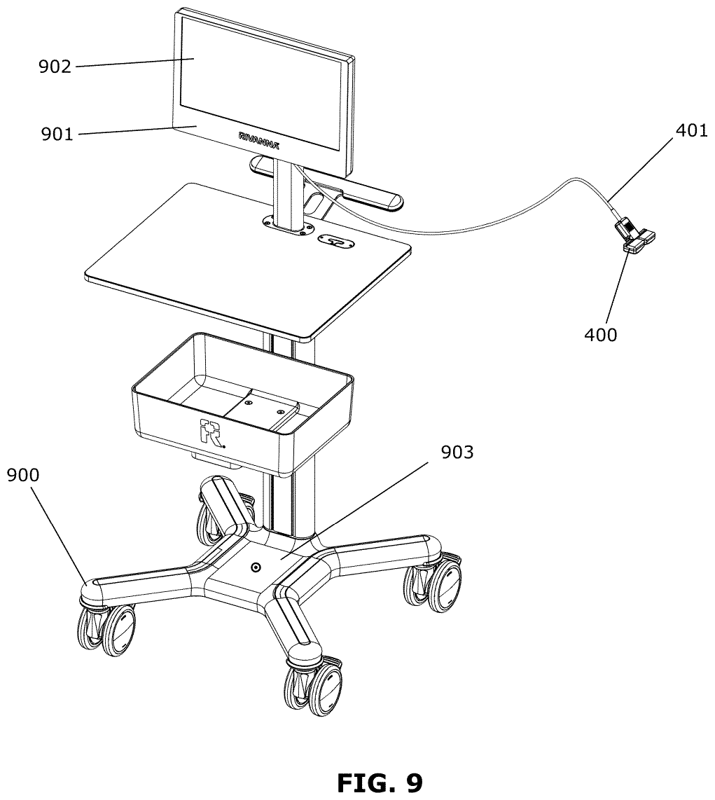

In an exemplary embodiment depicted in FIG. 9, the ultrasound probe 400 can be connected by an electrical signal cable 401 to a mobile cart 900 to allow the imaging device to be moved to the bedside and positioned at the required or desired orientation for acquiring images of the patient's anatomy. The cart 900 can include an enclosure 901 which may contain a computer processor and monitor 902, battery 903, and other associated electronics familiar to those skilled in the art which are needed to power and communicate with the imaging device 400. The cart 900 can be outfitted with additional input/output devices such as a keyboard, mouse, or monitor 902, which may also be a touchscreen display. The monitor 902 may be positionally adjustable about the cart in order to orient the imaging device 400 and monitor in various relative positions for the needle guidance procedure. In a preferred embodiment, the enclosure 901 may simultaneously contain the monitor 902, the computer processor, and the ultrasound front-end electronics. A computer processor within the enclosure 901 may be used to perform ultrasound signal and image processing steps required to form an ultrasound image reconstruction that can be displayed on the monitor 602. Such processing steps are known to those skilled in the art of medical ultrasound and may include but are not limited to: beamforming, bandpass filtering, scan conversion, and image rendering. Two and three-dimensional images may be rendered using various techniques, including simultaneous display, as described in Mauldin et al., U.S. Pat. No. 11,504,095 (incorporated herein by reference). In a preferred embodiment, the computer processor in the enclosure 601 can receive signals from the ultrasound probe 400 indicating the position of the probe in the apparatus, which can be used for interpreting spatial position of the real-time image data acquired. In a preferred embodiment, the registration of spatial position of the imaging data can be used to reconstruct 3-dimensional ultrasound images that are functionally equivalent to fluoroscopic imaging of skeletal anatomy.

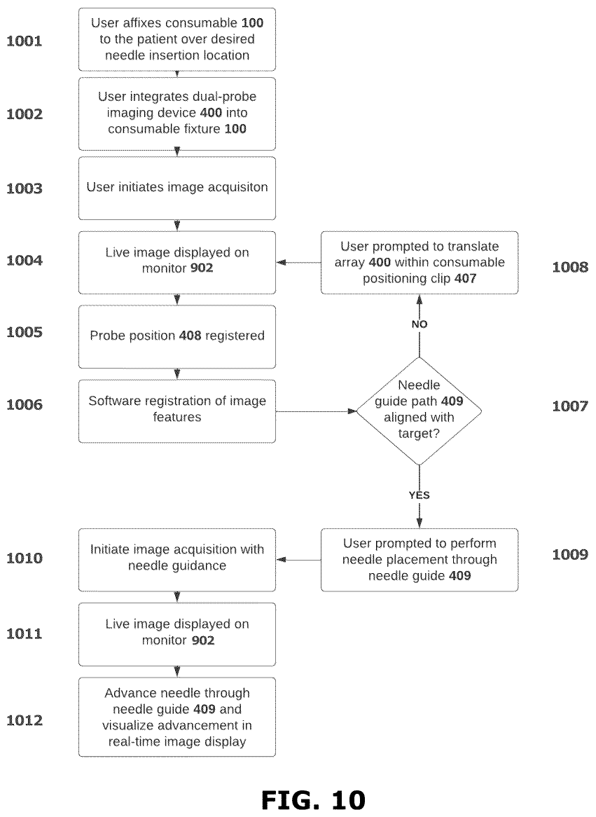

A flow diagram of an exemplary embodiment for the present invention used in a clinical needle guidance procedure is depicted in FIG. 10. At block 1001, the user first affixes the apparatus base component 100 to the patient over the desired needle insertion location. At block 1002 the ultrasound probe is integrated into the apparatus as depicted in FIG. 4 at the desired imaging position and coupled to the patient's body using ultrasound coupling gel or other lubricant compatible with medical ultrasound and known to those skilled in the art. Next, at block 1003 an image acquisition is initiated by the user. Initiation may be achieved through the monitor 902, user interface buttons, or by other means known to those of skill in the art. At block 1004, the ultrasound image acquisitions are transmitted to the computer system within the enclosure 901 and the reconstructed image is displayed on the monitor 902. In an embodiment where the ultrasound probe 400 is contained within the position tracking clip 407 illustrated in FIG. 4C, the user may translate the probe manually along the position tracking clip to acquire 3-dimensional images. In the same embodiment, in block 1005 the position of the ultrasound probe 400 is registered during image acquisition in order to build a 3-dimensional image volume. In block 1006, the live image is registered against a representation of other imaging anatomy for the procedure, such as an ideal imaging anatomy for the procedure, which may be derived from 3-dimensional ultrasound data or other representations of the anatomy. In block 1007 a determination is made whether the current image meets criteria indicating the needle guide is aligned with the needle injection target. This assessment may be produced automatically by a processing algorithm run on the computer system, such as described by Mauldin et al., U.S. Pat. No. 11,504,095 (incorporated herein by reference), or it may be achieved by the user through visual assessment of the rendered imaging results. If these criteria are not met, block 1008 guides the user to adjust position of the ultrasound probe 400 to provide better alignment with the target anatomy, providing guidance to address challenges that may be encountered in carrying out the procedure. If the criteria are met, block 1009 guides the user to proceed to needle placement. In aspects, user initiation of the needle guidance mode 1010 results in a transition in software to an imaging mode that enhances real-time visualization of the needle during insertion 1011. Finally, at block 1012, the user advances the needle through the needle guide 409 and the needle is visualized within the ultrasound image rendering displayed on the monitor 901 as it advances to the needle target.

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1) A system for affixing an ultrasound transducer to a patient, the system comprising:
one or more base component capable of being removeably affixed to the patient near or adjacent to a patient anatomy; and
one or more securing component interfacing with the ultrasound transducer and the one or more base component to (a) attach the ultrasound transducer to the one or more base component near or adjacent to the patient anatomy and (b) substantially maintain acoustic coupling of said ultrasound transducer and the patient anatomy;
wherein the system allows a user to access between about 10% and about 95% of the patient anatomy near or adjacent to the ultrasound transducer for insertion of a needle, a probe, or an injectable device.

2) The system for affixing an ultrasound transducer to a patient of claim 1, further comprising a mechanism for adjusting a position of the ultrasound transducer that allows for moving the ultrasound transducer at least one of: up-and-down, left-and-right, diagonally across a patient contact surface, and at an angle relative to the patient contact surface.

3) The system for affixing an ultrasound transducer to a patient of claim 2, wherein the mechanism for adjusting a position of the ultrasound transducer allows for adjusting an angulation of the ultrasound transducer relative to the patient anatomy.

4) The system for affixing an ultrasound transducer to a patient of claim 2, wherein the mechanism for adjusting a position of the ultrasound transducer comprises a member capable being manipulated by the user to substantially maintain an ultrasound transducer angulation relative to the patient anatomy, or optionally to set an ultrasound transducer angulation relative to the patient anatomy.

5) The system for affixing an ultrasound transducer to a patient of claim 1, wherein the one or more securing component comprises one or more of a band, a clip, a track, an adhesive, or a case.

6) The system for affixing an ultrasound transducer to a patient of claim 5, wherein the track comprises at least two elongated members extending between two base components of the one or more base component, wherein the at least two elongated members are positioned substantially parallel to one another, and wherein the at least two elongated members attach to the ultrasound transducer and allow the ultrasound transducer to slide along the track between the two base components of the one or more base component.

7) The system for affixing an ultrasound transducer to a patient of claim 1, wherein the one or more securing component comprises an acoustically transmissive member between (a) the ultrasound transducer or a probe sheath and (b) a patient contact surface.

8) The system for affixing an ultrasound transducer to a patient of claim 7, wherein the acoustically transmissive member is comprised of an adhesive film that temporarily adheres the ultrasound transducer or the probe sheath to the patient contact surface for the purpose of gel-free scanning.

9) The system for affixing an ultrasound transducer to a patient of claim 1, wherein the one or more securing component is physically integrated with a probe sheath or a drape.

10) The system for affixing an ultrasound transducer to a patient of claim 9, wherein the probe sheath or the drape completely or partially cover the ultrasound transducer to substantially maintain a sterile field.

11) The system for affixing an ultrasound transducer to a patient of claim 1, wherein the one or more securing component comprises an acoustically transmissive component between the ultrasound transducer and a probe sheath or a drape.

12) The system for affixing an ultrasound transducer to a patient of claim 1, wherein the one or more securing component comprises an acoustic coupling dispensation component.

13) The system for affixing an ultrasound transducer to a patient of claim 12, wherein the acoustic coupling dispensation component comprises one or more of an absorptive pad, a sponge, or an encapsulated reservoir, and wherein the acoustic coupling dispensation component dispenses acoustic coupling fluid to substantially maintain acoustic contact between the ultrasound transducer and the patient anatomy.

14) The system for affixing an ultrasound transducer to a patient of claim 12, wherein the acoustic coupling dispensation component is completely or partially saturated with water, saline, betadine, or acoustic gel.

15) The system for affixing an ultrasound transducer to a patient of claim 12, wherein the acoustic dispensation component applies acoustic coupling fluid along an outer surface of a probe sheath to substantially maintain acoustic contact between the probe sheath and the patient anatomy.

16) The system for affixing an ultrasound transducer to a patient of claim 12, wherein the acoustic dispensation component applies acoustic coupling fluid along an inner surface of a probe sheath to substantially maintain acoustic contact between the ultrasound transducer and the probe sheath.

17) The system for affixing an ultrasound transducer to a patient of claim 1, wherein the one or more securing component provides an aperture, an opening, or a slot, configured to receive the needle, the probe, or the injectable device, wherein the aperture, the opening, or the slot, substantially aligns or guides a percutaneous injection of the needle, the probe, or the injectable device.

18) The system for affixing an ultrasound transducer to a patient of claim 1, wherein the one or more base component further comprises or is attached to a drape for covering all or a portion of a patient's body nearby or adjacent to a location where the needle, the probe, or the injectable device is to be inserted into the patient.

19) The system for affixing an ultrasound transducer to a patient of claim 1, further comprising a cable connected to the ultrasound transducer, wherein the cable is oriented relative to one or more of the ultrasound transducer and the one or more base component, to at least one of minimize a vertical profile of the ultrasound transducer relative to the patient anatomy, stabilize one or more of the ultrasound transducer and the one or more base component, and maximize procedure access around a base of the ultrasound transducer.

20) The system for affixing an ultrasound transducer to a patient of claim 1, further comprising a cable connected to the ultrasound transducer, wherein the cable is oriented relative to one or more of the ultrasound transducer and the one or more base component such that the ultrasound transducer-cable interface is angled between about 30 degrees and about 90 degrees away from a body of the ultrasound transducer.

21) The system for affixing an ultrasound transducer to a patient of claim 1, further comprising a cable connected to the ultrasound transducer, wherein the cable is oriented relative to one or more of the ultrasound transducer and the one or more base component, such that the ultrasound transducer-cable interface is centered or off-centered along a front or a back of a body of the ultrasound transducer.

22) The system for affixing an ultrasound transducer to a patient of claim 1, further comprising a cable connected to the ultrasound transducer, wherein the cable is oriented relative to one or more of the ultrasound transducer and the one or more base component, such that the ultrasound transducer-cable interface is centered or off-centered along one or more sides of a body of the ultrasound transducer.

23) The system for affixing an ultrasound transducer to a patient of claim 1, further comprising one or more sensor for measuring an absolute or a relative orientation of the ultrasound transducer relative to at least one of the one or more base component, the one or more securing component, or the patient anatomy.

24) The system for affixing an ultrasound transducer to a patient of claim 23, wherein at least one of the one or more sensor is disposed within a housing of the ultrasound transducer, and wherein at least one of the one or more sensor is disposed within at least one of the one or more base component or the one or more securing component.

25) The system for affixing an ultrasound transducer to a patient of claim 1, wherein the ultrasound transducer is an ultrasound-based imaging dual-array probe comprising two ultrasound transducer arrays.

26) The system for affixing an ultrasound transducer to a patient of claim 1, wherein the ultrasound transducer is an ultrasound-based imaging dual-array probe comprising two ultrasound transducer arrays and a longitudinal slot, the two ultrasound transducer arrays disposed on opposing sides of the longitudinal slot.

27) The system for affixing an ultrasound transducer to a patient of claim 1, wherein an orientation of the ultrasound transducer and two-dimensional ultrasound image data is processed by a computer processor to form a volumetric three-dimensional ultrasound dataset to formulate a visualization of the patient anatomy located nearby or adjacent to an insertion cavity of the patient.

28) The system for affixing an ultrasound transducer to a patient of claim 27, wherein the orientation of the ultrasound transducer is used by the computer processor to relate a location of a two-dimensional ultrasound scan plane to the user relative to an anatomical reference comprised of at least one of a volumetric ultrasound dataset or a volumetric reference model of the patient anatomy.

29) The system for affixing an ultrasound transducer to a patient of claim 1, wherein the ultrasound transducer is an ultrasound-based imaging dual-array probe with dual-arrays disposed on opposing sides of a longitudinal slot, and wherein the longitudinal slot and imaging generated by the dual-arrays provide in-plane guidance for insertion of the needle, the probe, or the injectable device, into an insertion cavity of the patient at a desired anatomical location.

30) The system for affixing an ultrasound transducer to a patient of claim 1, wherein the one or more base component comprises one or more strap disposed around a body of the patient.

31) The system for affixing an ultrasound transducer to a patient of claim 30, wherein the ultrasound transducer is capable of accepting the one or more strap.

32) The system for affixing an ultrasound transducer to a patient of claim 1, wherein the one or more base component comprises or is integrated into a procedural drape.
